(19) United States
(12) Patent Application Publication
(10) Pub. No.: US 2011/0270212 A1
(43) Pub. Date: Nov. 3, 2011

(54) PHARMACOKINETIC CONTROL FOR OPTIMIZED INTERFERON DELIVERY

(75) Inventors: William P. Van Antwerp, Valencia, CA (US); Thomas J. Vasicek, Hopkins, MN (US)

(73) Assignee: MEDTRONIC, INC., Minneapolis, MN (US)

(21) Appl. No.: 12/681,751

(22) PCT Filed: Oct. 3, 2008
(86) PCT No.: PCT/US2008/078843

§ 371 Date: Apr. 5, 2010

Related U.S. Application Data
(60) Provisional application No. 60/997,897, filed on Oct. 5, 2007.

(51) Int. Cl. A61K 38/21; A61M 5/142; A61P 31/14

(52) U.S. Cl. 604/500; 424/85.7

(57) ABSTRACT

Methods and devices for treating patients having chronic hepatitis C infection so as to eradicate detectable HCV-RNA and/or inhibit the emergence of a drug resistant HCV variant are disclosed. Certain methods of the invention involve the use of a continuous infusion pump in a multiphasic combination therapy using a therapeutically effective amount of a small molecule inhibitor such as ribavirin and a therapeutically effective amount of interferon-α.

### REFERENCE TO RELATED APPLICATIONS

This application claims priority under Section 119(e) from U.S. Provisional Application Ser. No. 60/997,897 filed Oct. 5, 2007, the contents of which are incorporated herein by reference.

### FIELD OF THE INVENTION

The invention relates to methods and devices for treating viral infections such as hepatitis C infections with the combination of small molecule inhibitors such as ribavirin and cytokines such as interferon-α.

### BACKGROUND OF THE INVENTION

Hepatitis C virus (HCV) infection is the most common chronic blood borne infection in the United States. Chronic liver disease is the tenth leading cause of death among adults in the United States, accounting for approximately 25,000 deaths annually, or approximately 1% of all deaths. The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million.

Currently, treatments for chronic hepatitis C infection typically include the administration of combinations of ribavirin and interferon-α. Ribavirin is a nucleoside analog that when incorporated into cells, interferes with viral replication (similar to action of AZT in HIV infection). It is interesting to note that while ribavirin is not effective as a stand-alone therapy for HCV, it potentiates interferon effectiveness through an as yet unknown mechanism. For example, in controlled clinical studies, ribavirin monotherapy has negligible efficacy and PEG-interferon alone has an effectiveness of 11% in a genotype 1 population. However, when ribavirin is combined with interferon-α, the therapeutic effectiveness of the combination is 29% in this population (see, e.g. Sjogren et al., Dig Dis Sci. 2005 April; 50(4):727-32, the contents of which are incorporated by reference). A variety of such therapeutic methods for the treatment of hepatitis C infection are described for example in PCT patent applications such as WO 2005/067454; WO2005/018330; WO2005/062949; WO2006/130553; WO20060130626; and WO2006/130627; United States patent applications such as 2005/0191275; US 2005/0201980; 2007/004635; US2006/281689; and 2006/276405 and articles such as Perdita, et. al., World Journal of Gastroenerology, 7(2):222-227, (April 2001); Bizollon, et. al., Hepatology, 26(2):500-504, (August 1997); Alberti, et. al. Liver Transplantation, 7(10):870-876, (October 2001); Shakil, et. al., Hepatology, 36(5):1253-1258, (November 2002); Schalm, et. al., Gut, 46:562-568, (April 2000); and Yurdaydin et al., Journal of Viral Hepatitis, 12(7):262-268, (May 2005), the contents of which are incorporated herein by reference. Unfortunately however, while great strides have been made in the treatment of HCV infection, clinical success rates have progressed slowly since the introduction of interferon into the clinic as shown in FIG. 1 (see, e.g. Smith, R., Nat Rev Drug Discov. 2006, 5(9):715-6, the contents of which are incorporated by reference).

The pharmacokinetics of a number of alpha interferons approved for the treatment of HCV infection is shown in FIG. 2 and FIG. 3. As shown in the FIG. 2, Pegasys takes several weeks to reach a peak concentration and concomitantly takes several weeks to clear in case of an overdose. As shown in FIG. 3, Peg-Intron by comparison peaks at about 1 to 2 days but by day 4 or 5 after a weekly injection, the plasma concentration is essentially zero. The soluble interferons by comparison have very short half lives of 1.5 hours and, when given three times a week, there are considerable times during the course of a week when there is no drug on board at all. This is made clear in FIG. 3 which shows the interferon plasma levels during the first week of therapy that is most critical when dosing with very potent oral enzyme inhibitors.

Based on the current clinical practice success rate of less than 50% for genotype 1 or 1a patients, new therapies are highly desirable. Therapies currently in clinical development include those based on combining interferon therapy with either protease inhibitors such as those that inhibits the activity of the HCV serine protease NS3-4A or polymerase inhibitors such as those that inhibits the activity of HCV non-structural protein 5B (NS5B) polymerase (see, e.g. Lin et al., Infect Disord Drug Targets. 2006 March; 6(1):3-16; and Koev et al., Antiviral Res. 2007 January; 73(1):78-83. Epub 2006, the contents of which are incorporated by reference). Polymerase inhibitors can be either nucleoside analogs or non-nucleoside analog inhibitors (see, e.g. Beaulieu, Curr Opin Drug Discov Devel. 2006 September; 9(5):618-26, the contents of which are incorporated by reference).

Small molecule enzyme inhibitors while very potent, can lead to the emergence of HCV resistant virions like their anti-HIV counterparts (see, e.g. Neyts, Antiviral Res. 2006 September; 71(2-3):363-71. Epub 2006, the contents of which are incorporated by reference). Therapeutic methods that utilize combinations of an interferon-α and ribavirin, when continued over the necessary long periods of time, can produce HCV variants that are resistant to one or both of these therapeutic agents. As is known in the art, the emergence of such drug resistant pathogens is a serious concern for the medical community and public health. In addition, the pharmacokinetics of current interferon therapies are not well tuned to the requirements of combination therapy where interferon-α is used in conjunction with either a protease or polymerase inhibitor. Accordingly, there is a need for improved methods for treating viral infections such as hepatitis C, in particular the development of methods which are designed to treat HCV infected individuals while simultaneously avoiding the conditions under which drug resistant variants can emerge in order to inhibit the development of such variants.

### SUMMARY OF THE INVENTION

The invention disclosed herein provides methods and devices for administering combinations of therapeutic agents while precisely tailoring the specific dose, timing and means of administration of each agent in order to optimize a clinical outcome. Typical embodiments of the invention include a design predicated on the pharmacokinetics of one or more interferon-α species and of small antiviral molecules such as ribavirin. These embodiments include for example a three phase treatment regimen for individuals infected with Hepatitis C virus. In one such embodiment of the invention, a first induction phase (typically day 0→day 4˜11, depending upon observed changes in the viral load) comprises the administration of high doses of oral ribavirin and subcutaneous interferon-α (typically by continuous pumping using a medication infusion pump). This induction phase is designed to clear free virus in the blood and block denovo hepatocyte infection. After the induction phase, any viral mutants having arisen during the induction phase due to small molecule exposure (e.g. ribavirin resistant viral mutants) will have an impaired fitness as compared to the wild-type virus and consequently have enhanced interferon sensitivity (see, e.g. Tong et al., Antiviral Res. 2006, 70(2):28-38). A second antiviral phase is then initiated which comprises the administration of low doses of ribavirin in the absence of interferon-α (typically days 5˜11 or to some specific end point such as a predetermined viral load for example less than 100 copies of HCV-RNA per milliliter of serum). This antiviral phase is designed to eliminate HCV in infected hepatocytes without resulting in combined ribavirin/interferon resistance. A third final phase (typically 12 or more weeks) is then initiated which comprises the administration of oral ribavirin in combination with subcutaneous interferon-α (typically by continuous pumping using a medication infusion pump). This final phase is designed to target any remaining virions that are resistant to the small molecule inhibitor such as ribavirin, but due to the intervening antiviral phase, are not interferon-α resistant. In certain embodiments of the invention, at least one additional HCV small molecule inhibitor antiviral compound is administered during one or more of the sequential phases (e.g. the antiviral phase), for example VX-950, SCH 503034, R1626 or R71278. Typically during these phases, the HCV viral burden is monitored via a method such as rt-PCR and/or viral genotyping so that the timing and/or dose of each agent in a phase can be appropriately adjusting in accordance with observed changes in the viral burden. Such embodiments of the invention are designed to treat individuals while simultaneously reducing the emergence of HCV strains that are resistant to small molecule inhibitors such as ribavirin and/or interferon-α.

A typical embodiment of the invention is a method for treating a hepatitis C virus infected patient with a combination of interferon-α and ribavirin, the method comprising administering the interferon-α and ribavirin in sequential phases comprising: an induction phase which comprises co-administering to the patient a high dose of ribavirin and a high dose of interferon-α for at least 4 days, wherein the period of time of co-administration in the induction phase is sufficient to reduce the concentration of hepatitis C virus to a predetermined level, for example less than less than 100 copies of HCV-RNA per ml of serum; an antiviral phase which comprises administering to the patient a low dose of ribavirin in the absence of interferon-α for at least 5 days; and a final phase which comprises co-administering to the patient a therapeutically effective amount of ribavirin and interferon-α for at least 12 weeks, wherein the period of time of co-administration in the final phase is sufficient to reduce the concentration of hepatitis C virus to a predetermined level, for example less than 100 copies of HCV-RNA per milliliter of serum. Certain embodiments of the invention further comprise the step of administering to the patient at least one additional HCV antiviral compound during one or more of the sequential phases (e.g. the antiviral phase), for example other small molecule inhibitors such as VX-950, SCH 503034, R1626 or R71278. In typical embodiments of this method, the interferon-α or the small molecule inhibitors such as ribavirin in at least one of the sequential phases is administered using a continuous infusion pump. Optionally for example, the interferon-α is administered using a continuous infusion pump during at least one of the induction or the final phases, while the small molecule inhibitors such as ribavirin is administered orally during at least one of the sequential phases of this method.

The methods of the invention can be practiced on a wide variety of individuals infected with HCV including those previously treated for HCV infection or having a specific HCV strain. For example, some embodiments of the invention include the step of selecting the patient for treatment by identifying them as one previously treated with a course of interferon-α therapy, wherein the previous course interferon-α therapy was observed to be ineffective to treat one or more symptoms associated with the HCV infection. Other embodiments of the invention include the step of selecting the patient for treatment by identifying the patient as one infected with HCV having a specific HCV genotype or subtype, for example Genotype 1 or Genotype 1a.

As methods which use combinations of interferon-α and ribavirin in the treatment of HCV infection are well known in the art, any one of a wide variety of therapeutically effective dosage regimens for these agents can be used or adapted for use in various embodiments of the invention. In certain embodiments of the invention for example, a high dose of ribavirin is used in the induction phase, one which comprises a dose of at least 1000 mg/day. Similarly, in certain embodiments of the invention for example, a high dose of interferon is used in the induction phase one which comprises a dose of at least 6 IU/day. In some embodiments of the invention, a low dose of ribavirin is used in the antiviral phase, one which comprises a dose of less than 400 mg/day. In certain embodiments of the invention, the dose of ribavirin in the final phase comprises at least 1000 mg/day while the dose of interferon in the final phase comprises at least 6 IU/day. As is known in the art, various species of interferon-α (for example interferon-α conjugated to a polyol) can be administered in methods designed to treat HCV infection. In certain embodiments of the invention, an interferon-α administered in one or more of the sequential phases is not conjugated to a polyol. In alternative embodiments of the invention an interferon-α administered in one or more of the sequential phases is conjugated to a polyol. In some embodiments of the invention, the interferon-α comprises two interferon-α species: a first interferon-α species that is conjugated to a polyol; and a second interferon-α species that is not conjugated to a polyol. Optionally different species of interferon-α are administered in one or more of the different sequential phases of the invention.

In certain embodiments of the invention, the status of HCV in the individual is monitored during one or more of the phases of the method to obtain information useful for tailoring the therapeutic regimen to the responsiveness of a specific individual. In some embodiments of the invention, the initial and then changing concentrations of hepatitis C virus in the serum of the patient is measured by a quantitative PCR method that is employed during the various phases of the method. In one illustrative embodiment, the status of HCV in the individual is monitored to determine if the period of time of co-administration in the induction phase or the final phase is sufficient to reduce the concentration of hepatitis C virus to a specific end point, for example less than 100 copies of HCV-RNA per ml of serum. In another illustrative embodiment, the status of HCV in the individual is monitored to determine if the dose of interferon-α or ribavirin or other small molecule inhibitor administered in a phase of the method is sufficient to reduce the concentration of hepatitis C virus to the same or yet another specific end point, for example less than 50 copies of HCV-RNA per ml of serum. In yet another illustrative embodiment, the status of HCV in the individual is monitored to determine if the duration of the administration of interferon-α or the ribavirin during a phase of the method is sufficient to reduce the concentration of hepatitis C virus to a yet another specific end point, for example below levels of detectability in serum. Similar embodiments of the invention include the step of testing the treated patient during a phase of the method for the presence of an HCV variant that exhibits resistance to interferon-α or ribavirin (or both interferon-α and ribavirin).

Related embodiments of the invention include methods of inhibiting the emergence of a hepatitis C virus (HCV) having resistance to interferon-α and/or small molecule inhibitors such as ribavirin in a patient infected with wild-type hepatitis C virus and being treated with a combination of interferon-α and ribavirin. Typical embodiments comprise administering interferon-α and ribavirin in sequential phases comprising: an induction phase which comprises co-administering to the patient a high dose of ribavirin and a high dose of interferon-α for at least 4 days, wherein the period of time of co-administration in the induction phase is sufficient to reduce the concentration of hepatitis C virus to less than 100 copies of HCV-RNA per ml of serum from the patient as measured by a quantitative PCR method; an antiviral phase which comprises administering to the patient a low dose of ribavirin in the absence of interferon-α for at least 5 days; and a final phase which comprises co-administering to the patient a high dose of ribavirin and a high dose of interferon-α for at least 12 weeks, wherein the period of time of co-administration in the final phase is sufficient to reduce the concentration of hepatitis C virus to less than 100 copies of HCV-RNA per ml of serum from the patient as measured by a quantitative PCR method so that emergence of a hepatitis C virus (HCV) having resistance to interferon-α and ribavirin in the patient infected with wild-type hepatitis C virus is inhibited. Certain embodiments of the invention further comprise the step of administering to the patient at least one additional HCV antiviral compound during one or more of the sequential phases, for example VX-950, SCH 503034, R1626 or R71278 and then monitoring the viral load in the patient by a quantitative PCR method.

Optionally in such methods, the interferon-α, ribavirin or other antiviral compound in at least one of the sequential phases is administered using a continuous infusion pump. In certain embodiments of this method, the interferon-α administered in at least one of the phases comprises two interferon-α species: a first interferon-α species that is conjugated to a polyol; and a second interferon-α species that is not conjugated to a polyol. Optionally in such methods, the patient is identified prior to administration of interferon-α or ribavirin as one infected with HCV having Genotype 1 or Genotype 1a. Typically the duration of administration or the dose of interferon-α or ribavirin during a phase of this method is adjusted based upon a determination of HCV-RNA copy number per milliliter of serum. Certain embodiments of these methods include the step of testing the treated patient during a phase of the method for the presence of an HCV variant that exhibits resistance to interferon-α or ribavirin or both interferon and ribavirin, for example by genotyping or viral polynucleotide sequencing methods.

Other objects, features and advantages of the present invention will become apparent to those skilled in the art from the following detailed description. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE FIGURES

FIG. 1 is a bar graph showing the success of interferon based Hepatitis C therapies with various therapeutic agents across all HCV genotypes.

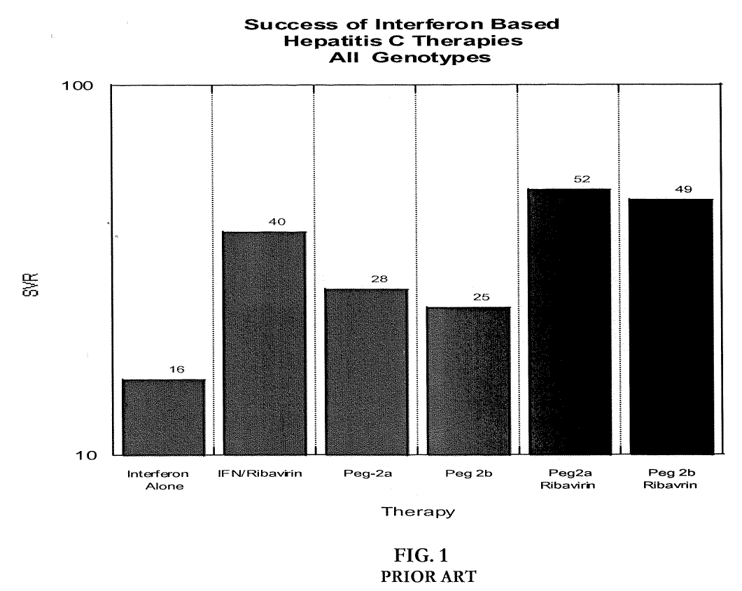

FIG. 2 is a graph showing the pharmacokinetics of various species of interferon-α over a period of 84 days.

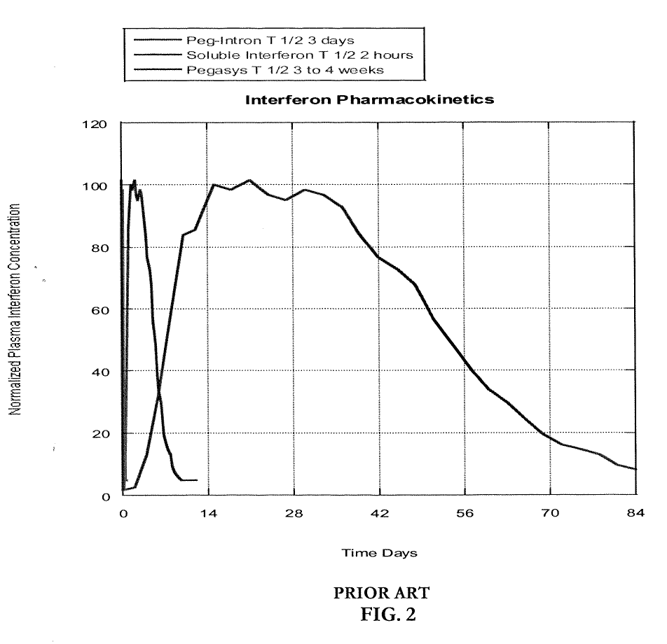

FIG. 3 is a graph showing the pharmacokinetics of various species of interferon-α over a period of 7 days.

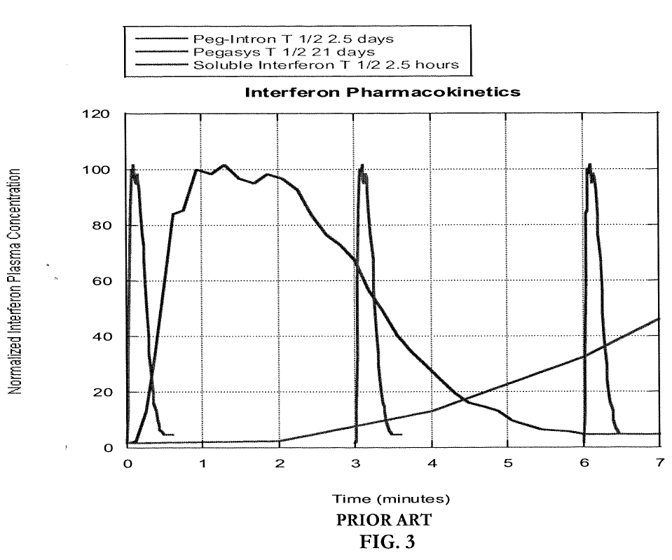

FIG. 4A-4D are graphs showing that plasma viral loads typically show a triphasic decay following interferon/ribavirin therapy. The first phase (which is very fast) corresponds to clearance of free virus in the blood of the patient, often from very high 10⁷ per ml levels. This phase also encompasses the blocking of de-novo infection of hepatocytes.

The second phase corresponds to the net loss of infected hepatocytes. This phase is often much slower than the first phase since interferon can only act on HCV that is replicating in the cells.

The third phase may be ascribed either to the immuno-modulatory effect of interferon on the non-replicating HCV in hepatocytes or alternatively to the potential mutagenic effects of ribavirin on the HCV particles.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art, such as, for example, the widely utilized molecular cloning methodologies described in Ausubel et al., Current Protocols in Molecular Biology, Wiley Interscience Publishers, (1995) and Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

### DEFINITIONS

The term “administer” means to introduce a therapeutic agent into the body of a patient in need thereof to treat a disease or condition.

The term “continuous infusion system” refers to a device for continuously administering a fluid to a patient parenterally for an extended period of time or for, intermittently administering a fluid to a patient parenterally over an extended period of time without having to establish a new site of administration each time the fluid is administered. The fluid typically contains a therapeutic agent or agents. The device typically has one or more reservoir(s) for storing the fluid(s) before it is infused, a pump, a catheter, cannula, or other tubing for connecting the reservoir to the administration site via the pump, and control elements to regulate the pump. The device may be constructed for implantation, usually subcutaneously. In such a case, the reservoir will usually be adapted for percutaneous refilling.

The term “treating” and/or “treatment” refers to the management and care of a patient having a pathology such as a viral infection or other condition for which administration of one or more therapeutic compounds is indicated for the purpose of combating or alleviating symptoms and complications of those conditions. Treating includes administering one or more formulations of the present invention to prevent the onset of the symptoms or complications, alleviating the symptoms or complications, or eliminating the disease, condition, or disorder. As used herein, “treatment” or “therapy” refer to both therapeutic treatment and prophylactic or preventative measures.

The term “therapeutically effective amount” refers to an amount of an agent (e.g. a cytokine such as interferon-α or small molecule inhibitors such as ribavirin) effective to treat at least one sign or symptom of a disease or disorder in a human. Amounts of an agent for administration may vary based upon the desired activity, the diseased state of the patient being treated, the dosage form, method of administration, patient factors such as the patient's sex, weight and age, the underlying causes of the condition or disease to be treated, the route of administration and bioavailability, the persistence of the administered agent in the body, the formulation, and the potency of the agent. It is recognized that a therapeutically effective amount is provided in a broad range of concentrations. Such range can be determined based on in vitro and/or in vivo assays.

The terms “continuous administration” and “continuous infusion” are used interchangeably herein and mean maintaining a steady state serum level of an agent such as interferon throughout the course of the treatment period. This can be accomplished by constantly or repeatedly injecting substantially identical amounts of interferon (typically with a continuos infusion pump device), e.g., at least every hour, 24 hours a day, seven days a week, such that a steady state serum level is achieved for the duration of treatment. Continuous interferon administration may be by subcutaneous or intravenous injection at appropriate intervals, e.g. at least hourly, for an appropriate period of time in an amount which will facilitate or promote in vivo inactivation of hepatitis C virus.

The term “patients or humans having hepatitis C infections” as used herein means any patient-including a pediatric patient-having hepatitis C and includes treatment-naive patients having hepatitis C infections and treatment-experienced patients having hepatitis C infections as well as those pediatric, treatment-naive and treatment-experienced patients having chronic hepatitis C infections. These patients having chronic hepatitis C include those who are infected with multiple HCV genotypes including type 1 as well as those infected with, inter alia, HCV genotype 2 and/or 3. The term “pediatric patient” as used herein means a patient below the age of 17, and normally includes those from birth to 16 years of age. The term “treatment-naive patients having hepatitis C infections” as used herein means patients with hepatitis C who have never been treated with ribavirin or any interferon, including but not limited to interferon-alpha, or pegylated interferon alpha. The term “treatment-experienced patients having hepatitis C infections” as used herein means patients with hepatitis C who have been treated with ribavirin or any interferon, including but not limited to interferon-alpha, or pegylated interferon alpha, including relapsers and non-responder. The term “patients having chronic hepatitis C infections” as used herein means any patient having chronic hepatitis C and includes “treatment-naive patients and treatment-experienced patients having chronic hepatitis C infections, including but not limited to relapsers and non-responders. The term “relapsers” as used herein means treatment-experienced patients with hepatitis C who have relapsed after initial response to previous treatment with interferon alone, or in combination with ribavirin. The term “non-responders” as used herein means treatment-experienced patients with hepatitis C who have not responded to prior treatment with any interferon alone, or in combination with ribavirin.

The term “wild-type Hepatitis C virus” is used herein according to its art accepted meaning and refers to the predominant genotypes of HCV that are found nature, in contrast to induced mutations (e.g. those mutant forms of HCV that are observed to be induced upon exposure to a small molecule such as ribavirin), or mutations generated via some other form of genetic manipulation (see, e.g. MacParland et al., Journal of General Virology (2006), 87, 3577-3586 and Kieffer et al., Hepatology, 2007, 46(3): 631-639, the contents of which are incorporated herein by reference).

As used herein, the term “synergy” or “synergism” or “synergistically” refers to the interaction of two or more agents so that their combined effect is greater than the sum of their individual effects.

The term “cytokine” is a generic term for a class of polypeptides released by cells that act as mediators of a wide variety of physiological processes. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; gonadotropin-associated peptide; inhibin; activin; vascular endothelial growth factor; integrin; thrombopoietin (TPO); nerve growth factors such as NGF-alpha; platelet-growth factor; transforming growth factors (TGFs) such as TGF-alpha and TGF-beta; insulin-like growth factor-I and -II; erythropoietin (EPO); osteoinductive factors; interferons such as interferon-alpha, -beta and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; a tumor necrosis factor such as TNF-alpha or TNF-beta; and other polypeptide factors including LIF and kit ligand (KL). The term “interferon” as used herein means the family of highly homologous species-specific proteins that inhibit viral replication and cellular proliferation and modulate immune response. Human interferons are grouped into three classes based on their cellular, origin and antigenicity: α-interferon (leukocytes), β-interferon (fibroblasts) and γ-interferon (T cells). Recombinant forms of each group have been developed and are commercially available. Subtypes in each group are based on antigenic/structural characteristics. A number of α-interferons (grouped into subtypes) having distinct amino acid sequences have been identified by isolating and sequencing DNA encoding these peptides. Both naturally occurring and recombinant α-interferons may be used in the practice of the invention. As used herein, the term cytokine includes proteins from natural sources or from recombinant cell culture and biologically active equivalents of the native sequence cytokines.

The term “antibody” when used for example in reference to an “antibody capable of binding HCV” is used in the broadest sense and specifically covers intact monoclonal antibodies, polyclonal antibodies, multispecific antibodies (e.g. bispecific antibodies) formed from at least two intact antibodies, and antibody fragments so long as retain their ability to immunospecifically recognize a target polypeptide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. In addition to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies, and is not to be construed as requiring production of the antibody by any particular method.

“Isolated,” when used to describe the various proteins disclosed herein, means protein that has been identified and separated and/or recovered from a component of its natural environment. Contaminant components of its natural environment are materials that would typically interfere with diagnostic or therapeutic uses for the protein, and may include enzymes, hormones, and other proteinaceous or non-proteinaceous solutes.

### Methods and Materials Associated with Embodiments of the Invention

Hepatitis C virus (HCV) is a positively stranded RNA virus that exists in at least six genetically distinct genotypes. These genotypes are designated Type 1, 2, 3, 4, 5 and 6, and their full length genomes have been reported (see, e.g. Genbank/EMBL accession numbers Type 1a: M62321, AF009606, AF011753, Type 1b: AF054250, D13558, L38318, U45476, D85516; Type 2b: D10988; Type 2c: D50409; Type 3a: AF046866; Type 3b: D49374; Type 4: WC-G6, WC-G11, WG29 (Li-Zhe Xu et al, J. Gen. Virol. 1994, 75: 2393-98), EG-21, EG-29, EG-33 (Simmonds et al, J. Gen. Virol. 1994, 74: 661-668), the contents of which are incorporated by reference). In addition, viruses in each genotype exist as differing “quasispecies” that exhibit minor genetic differences. The vast majority of infected individuals are infected with genotype 1, 2 or 3 HCV. HCV infection affects approximately 1.8% of the population in the USA and 3% of the population of the world. In over 85% of infected people, HCV causes a lifelong infection characterized by chronic hepatitis that varies in severity between individuals.

The currently therapy for the treatment of HCV infection is interferon-α, used alone or in combination with ribavirin. Such combination therapy can be highly effective for example in the treatment of HCV infection in patients previously treated with interferon alone and in patients never previously treated with interferon (see, e.g. Davis et al, NEJM, (1998), 339(21): 1493-99; Poynard et al, Lancet (1998) 352(9138): 1426-32, the contents of which are incorporated by reference).

Embodiments of the invention disclosed herein include optimized methods for using combinations or interferon-α and ribavirin in the treatment of HCV infection, methods which precisely control the timing, duration and/or dose of the interferon-α and ribavirin that is administered to a patient infected with HCV. In typical embodiments of the invention, the timing and dose of interferon and ribavirin delivery are tailored so as to control the pharmacokinetics in a manner that inhibits the development of interferon and/or ribavirin resistant HCV variants. Certain embodiments of the invention also administer to the patient at least one additional HCV antiviral compound, for example VX-950, SCH 503034, R1626 or R71278. Embodiments of the invention can be used to treat a variety of individuals suffering from chronic hepatitis C infection, including for example treatment failure patients, which include patients who failed to respond to previous HCV therapy (referred to as “non-responders”) or who initially responded to previous therapy, but in whom the therapeutic response was not maintained (referred to as “relapsers”).

A person suffering from chronic hepatitis C infection may exhibit one or more of the following signs or symptoms: (a) elevated serum alanine aminotransferase (ALT), (b) positive test for anti-HCV antibodies, (c) presence of HCV as demonstrated by a positive test for HCV-RNA, (d) clinical stigmata of chronic liver disease, (e) hepatocellular damage. Such criteria may not only be used to diagnose hepatitis C, but can be used to evaluate a patient's response to drug treatment. Elevated serum ALT and aspartate aminotransferase (AST) are known to occur in uncontrolled hepatitis C, and a complete response to treatment is generally defined as the normalization of these serum enzymes, particularly ALT (Davis et al., 1989, New Eng. J. Med. 321:1501-1506). ALT is an enzyme released when liver cells are destroyed and is symptomatic of HCV infection. Interferon causes synthesis of the enzyme 2′,5′-oligoadenylate synthetase (2′5′OAS), which in turn, results in the degradation of the viral mRNA. Houglum, 1983, Clinical Pharmacology 2:20-28. Increases in serum levels of the 2′5′OAS coincide with decrease in ALT levels. Histological examination of liver biopsy samples may be used as a second criteria for evaluation. See, e.g., Knodell et al., 1981, Hepatology 1:431-435, whose Histological Activity Index (portal inflammation, piecemeal or bridging necrosis, lobular injury and fibrosis) provides a scoring method for disease activity, the contents of which are incorporated by reference.

As discussed in detail below, certain embodiments of the invention include the step monitoring the HCV viral load in a subject and to adjust the therapeutic regimen based upon the observed result. Similarly, in certain embodiments of the invention, whether a particular method or methodological step is effective in combating an HCV infection can be determined by a number of factors, typically by measuring viral load. Alternatively, in certain circumstances, one can measure another parameter associated with HCV infection, including, but not limited to, liver fibrosis.

Viral load can be measured by a variety of procedures known in the art, for example by measuring the titer or level of virus in serum. These methods include, but are not limited to, a quantitative polymerase chain reaction (PCR) and/or a branched DNA (bDNA) test. Many such assays are available commercially, including a quantitative reverse transcription PCR(RT-PCR) (Amplicor HCV Monitor™ Roche Molecular Systems, New Jersey); and a branched DNA (deoxyribonucleic acid) signal amplification assay (Quantiplex™ HCV RNA Assay (bDNA), Chiron Corp., Emeryville, Calif.). See, e.g., Gretch et al. (1995) Ann. Intern. Med. 123:321-329. Illustrative assays used in embodiments of the invention to monitor viral titer in the methods of the invention include the COBAS Hepatitis C Virus (HCV) TaqMan Analyte-Specific Reagent Assay and/or the COBAS Amplicor HCV Monitor V2.0 and/or the Versant HCV bDNA 3.0 Assays (see, e.g. Konnick et al., Journal of Clinical Microbiology, May 2005, p. 2133-2140, Vol. 43, No. 5, the contents of which are incorporated by reference).

In certain embodiments of the invention, and HCV infected individual is administered a therapeutic agent such as interferon and/or a small molecule inhibitor such as ribavirin and the response to such agents is then observed by monitoring changes in the levels of HCV-RNA that are detectable in vivo, for example HCV-RNA copy number per milliliter of blood. In this context, an appropriate therapeutic response is associated with decreasing levels of HCV-RNA that are detectable in the blood of an infected individual. Ideally, a therapeutic regimen will reduce this number so that there is no longer any detectable HCV-RNA.

The term “no detectable HCV-RNA” in the context of the present invention means that there are fewer than 100 and typically fewer than 50 copies of HCV-RNA per ml of serum of the patient as measured by quantitative, multi-cycle reverse transcriptase PCR methodology. HCV-RNA is preferably measured in the present invention by research-based RT-PCR methodology well known to the skilled clinician. This methodology is referred to herein as HCV-RNA/qPCR. The lower limit of detection of HCV-RNA is typically 100 copies/mL. Serum HCV-RNA/qPCR testing and HCV genotype testing will be performed by a central laboratory. See also J. G. McHutchinson et al. (N. Engl. J. Med., 1998, 339:1485-1492), and G. L. Davis et al. (N. Engl. J. Med. 339:1493-1499, the contents of which are incorporated by reference).

While viral titers are the most important indicators of effectiveness of a dosing regimen, other parameters can also be measured as secondary indications of effectiveness. Secondary parameters include reduction of liver fibrosis; and reduction in serum levels of particular proteins. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20, the contents of which are incorporated by reference. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems. Another alternative but indirect method of determining viral load is by measuring the level of serum antibody to HCV. Methods of measuring serum antibody to HCV are standard in the art and include enzyme immunoassays, and recombinant immunoblot assays, both of which involve detection of antibody to HCV by contacting a serum sample with one or more HCV antigens, and detecting any antibody binding to the HCV antigens using an enzyme labeled secondary antibody (e.g., goat anti-human IgG). See, e.g., Weiss et al. (1995) Mayo Clin. Proc. 70:296-297; and Gretch (1997) Hepatology 26:43 S-47S, the contents of which are incorporated by reference.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase. Yet another secondary indicator of effectiveness of a treatment regimen is a change in the levels of serum alanine aminotransferase (ALT). Serum ALT levels are measured, using standard assays. In general, an ALT level of less than about 80, less than about 60, less than about 50, or about 40 international units per liter of serum is considered normal. In some embodiments, an effective amount of IFN-α is an amount effective to reduce ALT levels to less than about 200 IU, less than about 150 IU, less than about 125 IU, less than about 100 IU, less than about 90 IU, less than about 80 IU, less than about 60 IU, or less than about 40 IU.

Embodiments of the methods disclosed herein include the administration of interferon-α or “interferon-alpha” to an individual infected with HCV. Such embodiments of the invention optimize regimens for treating HCV infection using permutations of ribavirin and an interferon alpha treatments that are well known in the art, e.g., as disclosed in U.S. Pat. No. 6,299,872, U.S. Pat. No. 6,387,365, U.S. Pat. No. 6,172,046, U.S. Pat. No. 6,472,373, and U.S. Patent Application No. 200060257365, the disclosures of which are incorporated herein by reference. The term “interferon-alpha” as used herein means the family of highly homologous species-specific proteins that inhibit viral replication and cellular proliferation and modulate immune response. Typical suitable interferon-alphas include, but are not limited to, recombinant interferon alfa-2b such as Intron-A interferon available from Schering Corporation, Kenilworth, N.J., recombinant interferon alfa-2a such as Roferon interferon available from Hoffmann-La Roche, Nutley, N.J., recombinant interferon alpha-2c such as Berofor alpha 2 interferon available from Boehringer Ingelheim Pharmaceutical, Inc., Ridgefield, Conn., interferon alpha-n1, a purified blend of natural alpha interferons such as Sumiferon available from Sumitomo, Japan or as Wellferon interferon alpha-n1 (INS) available from the Glaxo-Welicome Ltd., London, Great Britain, or a consensus alpha interferon such as those described in U.S. Pat. Nos. 4,897,471 and 4,695,623 and the specific product available from Amgen, Inc., Newbury Park, Calif., or interferon alfa-n3 a mixture of natural alpha interferons made by Interferon Sciences and available from the Purdue Frederick Co., Norwalk, Conn., under the Alferon Tradename or recombinant interferon alpha available from Frauenhoffer Institute, Germany or that is available from Green Cross, South Korea. The use of interferon alfa-2a or alpha 2b is preferred. Since interferon alpha 2b, among all interferons, has the broadest approval throughout the world for treating chronic hepatitis C infection, it is most preferred. The manufacture of interferon alpha 2b is described in U.S. Pat. No. 4,530,901, the contents of which are incorporated by reference.

There are a number of commercially available pegylated and non-pegylated native, soluble interferon species that can be used in embodiments of the invention. For example, intron-a (interferon-α 2b, Schering Plough) was a first interferon approved for hepatitis C use. Intron-a is also indicated for a variety of cancer therapies including a list of hematological malignancies and hepatitis B. There is no mention of therapy failures in the Intron-a package insert, however the label for Intron-a plus ribavirin therapy is indicated only for naïve patients. The dosages for hepatitis C therapy are listed as 3 MU TIW followed by a 50% dose reduction if not tolerated well. Roferon (interferon-α 2a, Roche) is another interferon approved for hepatitis C. The indication list is almost identical and the dosage is the same, 3 MU TIW. There is no indication of use with ribavirin and no discussion of therapy failures in the package labeling. Infergen (interferon-αconsensus, Valeant) is labeled only for hepatitis C. Infergen is labeled as a 9 μg injection TIW in naïve patients and 15 μg TIW for patients who tolerated interferon but did not respond or relapsed. Peg-Intron (interferon-α 2b pegylated with a 12 kD PEG (polyethylene glycol), Schering Plough) was the first pegylated interferon introduced to the marketplace. Pegylation of the interferon leads to a molecule with reduced biological activity but a greatly increased circulating half-life in-vivo. Peg-Intron is labeled for weight based dosing with a single weekly injection in combination with ribavirin. Peg-intron is only labeled for naïve patients. The half-life of Peg-Intron is about 48 hours, so plasma levels of interferon are essentially zero by the end of day 7 following injection. Pegasys (interferon-α 2a pegylated with a 40 kD PEG, Roche) was the second pegylated interferon approved for clinical use. In contrast to Peg-Intron, Pegasys is typically delivered at the same dose for all patients; however the ribavirin component is typically dosed by weight. Like Peg-Intron, Pegasys is only indicated for interferon naïve patients. The pharmacokinetics of Pegasys are considerably different than Peg-intron due to the larger molecular weight of the PEG attached to the interferon. The circulating half-life of Pegasys is about 3 weeks, which might have considerable safety implications in the case of overdosing but does not allow for significantly reduced trough levels in the plasma. It is interesting to note that in both controlled clinical trials and in community practice, Peg-Intron and Pegasys therapies lead to very similar outcomes.

Certain embodiments of the methods disclosed herein include the administration of interferon-α that is conjugated to a polyol such as polyethylene glycol. Such interferon-α conjugates can be prepared by coupling an interferon alpha to a variety of water-soluble polymers. A non-limiting list of such polymers include polyethylene and polyalkylene oxide homopolymers such as polypropylene glycols, polyoxyethylenated polyols, copolymers thereof and block copolymers thereof. As an alternative to polyalkylene oxide-based polymers, effectively non-antigenic materials such as dextran, polyvinylpyrrolidones, polyacrylamides, polyvinyl alcohols, carbohydrate-based polymers and the like can be used. Such interferon alpha-polymer conjugates are described in U.S. Pat. No. 4,766,106, U.S. Pat. No. 4,917,888, European Patent Application No. 0 236 987, European Patent Application Nos. 0510 356, 0 593 868 and 0 809 996 (pegylated interferon alfa-2a) and International Publication No. WO 95/13090, the contents of which are incorporated by reference. As is known in the art, polyols for example can be conjugated to polypeptides at one or more amino acid residues, including lysine residues, as is disclosed in WO 93/00109. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to a polypeptide is termed “pegylation.” However, those skilled in the art recognize that other polyols, such as, for example, poly(propylene glycol) and polyethylene-polypropylene glycol copolymers, can be employed using the techniques for conjugation described herein for PEG. Illustrative examples of cytokines conjugated with PEG are shown, for example, in U.S. Pat. No. 5,795,569; U.S. Pat. No. 4,902,502; Wang et al., Biochemistry 2000, 39, 10634-10640; Leong et al., Cytokine 2001, 16(3): 24-36; and Kozlowski et al., BioDrugs 2001; 15(7): 419-429, the contents of which are incorporated by reference.

Typical embodiments of the invention use interferon-α that is conjugated to a polyethylene glycol polyol (i.e. a “pegylated interferon-alpha”). As used herein, the term “pegylated interferon-alpha” as used herein means polyethylene glycol modified conjugates of interferon-alpha, preferably pegylated interferon alfa-2a, pegylated interferon alfa-2b, or a pegylated consensus interferon, more preferably pegylated interferon alfa-2a and pegylated interferon alfa-2b. The preferred polyethylene-glycol-interferon alfa-2b conjugate is PEG₁₂₀₀₀-interferon alpha 2b. The phrases “12,000 molecular weight polyethylene glycol conjugated interferon alpha” and “PEG₁₂₀₀₀-IFN alpha” as used herein mean conjugates such as are prepared according to the methods of International Application No. WO 95/13090 and containing urethane linkages between the interferon alfa-2a or -2b amino groups and polyethylene glycol having an average molecular weight of 12000.

Embodiments of the methods disclosed herein include the administration of ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. The in vitro inhibitory concentrations of ribavirin are disclosed in Goodman & Gilman's “The Pharmacological Basis of Therapeutics”, Ninth Edition, (1996) McGraw Hill, New York, at pages 1214-1215. The Virazole product information discloses a dose of 20 mg/mL of Virazole aerosol for 18 hours exposure in the 1999 Physicians Desk Reference at pages 1382-1384. Typical ribavirin dosage and dosage regimens are also disclosed by Sidwell, R. W., et al. Pharmacol. Ther 1979 Vol 6. pp 123-146 in section 2.2 pp 126-130. Fernandes, H., et al., Eur. J. Epidemiol., 1986, Vol 2(1) ppl-14 at pages 4-9 disclose dosage and dosage regimens for oral, parenteral and aerosol administration of ribavirin in various preclinical and clinical studies.

In practicing the methods of the invention, the dosage amount, dosage period, dosage schedule, dosage route, and so on for interferon-α and/or ribavirin encompass those generally used in the art to administer these agents in a manner that produces an improvement in one or more physiological conditions associated with a chronic hepatitis C infection. In this context, skilled artisans understand that a variety of dosage regimens known in the art can be employed in and/or adapted to the methods of the invention (e.g. those described in United States Patent Applications 20060088502 and 20060024271 and U.S. Pat. No. 6,849,254 the contents of which are incorporated by reference).

As is known in the art, interferon-α can be administered at fairly high dose (e.g. up to 300 million IU/m² subcutaneously) without adverse reactions. In this context, medical personnel typically control and/or modify an interferon-α dosage regimen depending on the constellation of clinical factors observed in a specific individual (factors which are known to change during treatment). In particular, artisans understand that for HCV infections, one single predetermined regimen is not applicable to all patients and that optimally effective regimens are typically those that are individually designed in view of various factors observed in a specific individual. For example, medical personnel may select a specific interferon-α dosage regimen based upon the genotype or subtype of HCV that is observed to be infecting the patient and/or the amount of HCV-RNA per ml of serum in the patient as measured by a quantitative PCR method. As is similarly known in the art, the dosage regimen may be selected or controlled depending on the weight and age of a patient, whether the patient is known to be a nonresponder or relapser, or whether the patient is observed to have another pertinent pathological condition (e.g. cirrhosis of the liver, hepatocarcinoma, HIV infection, or the like). Depending upon for example the constellation clinical factors observed in a specific individual, the interferon-α (and ribavirin) can be administered on a weekly (QW), twice a week (BIW), three times a week (TIW), every other day (QOD) or on a daily basis. Similarly, depending upon for example the clinical factors and/or personal needs of the patients, these therapeutic agents can be administered via a variety of routes, for example subcutaneously, intramuscularly or intravenously. In certain dosage regimens, an infusion delivery device (e.g. a medication infusion pump) is used to deliver interferon-α. In other dosage regimens, an infusion delivery device is not used, and the interferon-α is delivered via injection with a conventional syringe. In this context, the following descriptions of various illustrative schemes for administering therapeutically effective amounts of the combination therapy of interferon-α and ribavirin are not limiting and are instead provided merely as typical examples of dosage regimens known in the art that can be employed and/or adapted to the methods of the invention.

In typical embodiments of the invention, the interferon-α administered is selected from one or more of interferon alpha-2a, interferon alpha-2b, a consensus interferon, a purified interferon alpha product (e.g. a purified interferon-α product produced by a recombinant technology) and/or a pegylated interferon-α. As is known in the art, an interferon-α dose can be characterized in international units (IU) or milligrams of polypeptide, optionally in the context of amount of agent per kilogram of patient weight and/or another measure of patient size (e.g. m²). In one illustrative embodiment of the invention, the interferon-α can be consensus interferon and the amount of interferon-α administered can be from 1 to 20 micrograms per week on a weekly (QW), twice a week (BIW), three times a week (TIW), every other day (QOD) or on a daily basis. Alternatively, the interferon-α administered can be a pegylated interferon alpha-2a and the amount of interferon-α administered is from 20 to 250 micrograms/kilogram per week on a weekly, TIW, QOD or daily basis. In another embodiment, the interferon-α administered can be a pegylated interferon alpha-2b and the amount of interferon-α administered can be from 0.5 to 2.0 micrograms per week on a QW, BIW, TIW, QOD, or on a daily basis. Optionally, the interferon-α can be selected from interferon alpha-2a, interferon alpha-2b, or a purified interferon-α product and the amount of interferon-α administered can be from 1 to 20 million IU per week on a daily to weekly basis. In one embodiment, the interferon-α administered is interferon-alpha-2b and the amount of interferon-α administered can be 2 to 10 (and typically 4, 5, 6, 7, or 8) million IU three to seven times a week. In such dosage regimes that are adapted to the methods of the invention, an infusion delivery device (e.g. a medication infusion pump) can be used to deliver interferon-α. Alternatively an infusion delivery device is not used in such dosage regimes, and the interferon-α is delivered via injection with a conventional syringe. While administration or infusion can be intermittent or continuous, the frequency of injection of the interferon composition will typically depend on the form of the composition. It will be understood that injection will be less frequent (e.g., once or twice a week) when using sustained release formulations or long-acting polymer conjugates (e.g. those conjugated with polyethylene glycol).

In certain embodiments when the interferon-α administered is selected from interferon alfa-2a, interferon alfa-2b, or a purified interferon-α product, the therapeutically effective induction dosing amount of interferon-α administered in the induction and/or final phases can be 6-10 MIU daily for a first specific time period (e.g. 2 weeks), followed by 3-5 MIU daily for another time period (e.g. 6 weeks), followed by 1-3 MIU daily for yet another time period (e.g. 16 weeks to 24 weeks). When the interferon-α administered is consensus interferon, the amount of consensus interferon administered in the first treatment period of twenty-four weeks can be from for example, 15 to 20 micrograms on a daily basis for two or more weeks, followed by 9 to 15 micrograms on a daily basis for twenty or more weeks. In such dosage regimes that are adapted to the methods of the invention, an infusion delivery device (e.g. a medication infusion pump) can be used to deliver interferon-α. Alternatively an infusion delivery device is not used in such dosage regimes, and the interferon-α is delivered via injection with a conventional syringe.

In certain embodiments where the pegylated interferon-α is a pegylated interferon alfa-2b, the therapeutically effective amount of pegylated interferon alfa-2b administered during a phase of the treatment can be the range of about 0.1 to 9.0 micrograms per kilogram of pegylated interferon alfa-2b administered per week, in single or divided doses, for example once a week or twice a week, typically in the range of about 0.1 to about 9.0 micrograms per kilogram of pegylated interferon alfa-2b administered once a week or can be in the range of about 0.05 to about 4.5 micrograms per kilogram of pegylated interferon alfa-2b administered twice a week, or can be in the range of about 0.5 to about 3.0 micrograms per kilogram of pegylated interferon alfa-2b administered per week, for example in the range of about 0.5 to about 3.0 micrograms per kilogram of pegylated interferon alfa-2b administered once a week or in the range of about 0.25 to about 1.5 micrograms per kilogram of pegylated interferon alfa-2b administered twice a week, or can be in the range of about 0.75 to about 1.5 micrograms per kilogram of pegylated interferon alfa-2b administered per week, typically in the range of about 0.75 to about 1.5 micrograms per kilogram of pegylated interferon alfa-2b administered once a week or about 0.375 to about 0.75 micrograms per kilogram of pegylated interferon alfa-2b administered twice a week. When the pegylated interferon-α administered as part of the combination therapy is a pegylated interferon alfa-2a, the therapeutically effective amount of pegylated interferon alfa-2a administered during the treatment in accordance with the present invention can be in the range of about 50 micrograms to about 500 micrograms once a week, for example about 180 micrograms to about 250 micrograms QW or the effective amount is in the range of about 50 micrograms to about 250 micrograms twice a week, for example about 90 micrograms to about 125 micrograms twice a week. In such dosage regimes that are adapted to the methods of the invention, an infusion delivery device (e.g. a medication infusion pump) can be used to deliver interferon-α. Alternatively an infusion delivery device is not used in such dosage regimes, and the interferon-α is delivered via injection with a conventional syringe.

In some embodiments where pegylated interferon-α is administered to pediatric patients as part of the methods of the invention is a pegylated interferon alfa-2b, the therapeutically effective amount of pegylated interferon alfa-2b administered during the treatment in accordance with the present invention can be in the range of about 0.1 to 9.0 micrograms per kilogram of pegylated interferon alfa-2b administered per week, in single or divided doses, optionally once a week or twice a week, typically about 0.1 to about 9.0 micrograms per kilogram of pegylated interferon alfa-2b administered once a week, or about 0.05 to about 4.5 micrograms per kilogram of pegylated interferon alfa-2b administered per week, in single or divided doses, optionally once a week or twice a week, for example from about 0.05 to about 4.5 micrograms per kilogram of pegylated interferon alfa-2b administered once a week, or optionally about 0.75 to about 3.0 micrograms per kilogram of pegylated interferon alfa-2b administered in single or divided doses, optionally once a week or twice a week, for example about 0.75 to about 3.0 micrograms per kilogram of pegylated interferon alfa-2b administered once a week or about 0.375 to about 1.5 micrograms per kilogram of pegylated interferon alfa-2b administered twice a week, and in certain embodiments about 2.25 to about 2.6 micrograms per kilogram of pegylated interferon alfa-2b administered once a week or about 1.1 to about 1.3 micrograms per kilogram of pegylated interferon alfa-2b administered twice a week. When the pegylated interferon-α administered to a pediatric patient is a pegylated interferon alfa-2a, the therapeutically effective amount of pegylated interferon alfa-2a administered during the treatment in accordance with the present invention can be in the range of about 50 micrograms to about 500 micrograms once a week, for example about 300 micrograms to about 375 micrograms QW or the therapeutically effective amount of pegylated interferon alfa-2a administered to a pediatric patient is in the range of about 50 micrograms to about 250 micrograms twice a week, optionally about 150 micrograms to about 190 micrograms once a week. In such dosage regimes that are adapted to the methods of the invention, an infusion delivery device (e.g. a medication infusion pump) can be used to deliver interferon-α. Alternatively an infusion delivery device is not used in such dosage regimes, and the interferon-α is delivered via injection with a conventional syringe.

In certain embodiments of the invention, interferon-α is administered at 1-20 million IU/m², for example daily, either intravenously, intramuscularly, or subcutaneously. Treatments with interferon-α at this range of doses and route of administration can last for example from about two weeks to six months or a year. In some embodiments of the invention, 2 to 15 (and typically is 4, 5, 6, 7, 8 or 9) million IU a day of an interferon-α is subcutaneously, intramuscularly or intravenously administered in a single dose or in divided doses every day or intermittently, for instance 2, 3, 4, 5, 6 or 7 times a week, for a period of 2 to 48 weeks or longer. In certain embodiments for example, in one or more of the phases of the invention an amount such as 6 to 15 million IU of interferon-α a day is administered every day for a first defined period, for example 2 to 8 weeks and then intermittently for a second defined period, for example, 22 to 46 weeks. As is known in the art, such a regimen, however, may appropriately be changed depending on the kind or dosage form of interferon-α. In the case of PEGylated interferon-α 2a (Pegasys, manufactured by Chugai Pharmaceutical Co., Ltd.) for example, it is generally possible to subcutaneously administer the interferon once a week, every time at a dose of for example 100-200 μg. In another illustrative embodiments of the invention, interferon-α is administered to the patient at 150 μg by subcutaneous injections. This treatment can typically last for four weeks or longer. In certain embodiments of the invention a recombinant interferon-α can be administered to a patient at doses of 0.01 to 2.5 mg/m² by intramuscular and/or intravenous bolus injections or alternating intramuscular and intravenous bolus injections with a minimum intervening period of 24, 48 or 72 hours. In such dosage regimes that are adapted to the methods of the invention, an infusion delivery device (e.g. a medication infusion pump) can be used to deliver interferon-α. Alternatively an infusion delivery device is not used in such dosage regimes, and the interferon-α is delivered via injection with a conventional syringe.

Ribavirin is administered as part of the combination therapy to the patient in association with pegylated interferon-α, that is, before, after or concurrently with the administration of the pegylated interferon-α. The pegylated interferon-α dose is typically administered during the same period of time that the patient receives doses of ribavirin. The amount of ribavirin administered concurrently with the pegylated interferon-α typically varies depending upon various factors such as a patient's weight and can be less than 399 mg per day in one or more phases of the invention (e.g. the antiviral phase) and also from about 400 to about 1600 mg per day, for example about 600 to about 1200 mg/day or about 800 to about 1200 mg day and typically about 1000 to about 1200 mg a day in another phase of the invention (e.g. the induction phase and/or final phase). In certain embodiments of the invention, the amount of ribavirin administered to a patient concurrently with the pegylated interferon-α can be for example from about 8 to about 15 mg per kilogram per day, typically about 8, 12 or 15 mg per kilogram per day, in divided doses. In such dosage regimes that are adapted to the methods of the invention, an infusion delivery device (e.g. a medication infusion pump) can be used to deliver interferon-α. Alternatively an infusion delivery device is not used in such dosage regimes, and the interferon-α is delivered via injection with a conventional syringe.

In certain embodiments of the invention, a high dose of interferon is continuously administered to a human patient exhibiting one of more of the above signs or symptoms in an amount and for a period of time sufficient to eliminate or at least alleviate one or more of the above-mentioned signs or symptoms. As noted above, the treatment regimen typically varies depending upon a specific patient's constellation of clinical symptoms. In certain embodiments and/or methodological steps of embodiments of the invention (e.g. the final phase), the duration of administration of a high dose of interferon is at least 4, 5, 6, 7, 8, 9, 10, 11 or 12 weeks or longer (e.g. 4-12 months). For treatment of chronic HCV with a high dose of interferon, a total daily dose of interferon (e.g. alpha interferon-2b) can for example range from 5-15 (and typically can be 6, 7, 8, 9 or 10) million IU per day.

In certain embodiments of the invention, a low dose of interferon-α is continuously administered to a patient exhibiting one of more signs or symptoms associated with HCV infection in an amount and for a period of time sufficient to eliminate or at least alleviate one or more of these signs or symptoms. As used herein, a low dose of interferon is typically an amount which for a given period of time is less than or equal to amounts used in traditional bolus or intermittent therapies over such a time period. In certain embodiments and/or methodological steps of embodiments of the invention (e.g. the induction and antiviral phases), the duration of administration is at least 4, 5, 6, 7, 8, 9, 10, 11 or 12 days or longer (e.g. 2, 3, 4, 5, or 6 weeks). In some embodiments and/or methodological steps of embodiments of the invention (e.g. the final phase), the duration of administration is at least is at least 4, 5, 6, 7, 8, 9, 10, 11 or 12 weeks or longer, for example 4-12 months. As noted above, the treatment regimen typically varies depending upon a specific patient's constellation of clinical symptoms. In certain embodiments for example for the treatment of chronic HCV with a low dose of interferon, a total weekly dose of interferon (e.g. alpha interferon-2b) can range from 2 to 15 million IU, or alternatively 5-10 million IU or 8-10 million IU per week and can be administered for example daily, every other day, or three times a week.

Certain embodiments of the invention use a low dose of ribavirin. As ribavirin is metabolized and cleared from the body by the liver, administration of ribavirin at a low-dose and/or as a slow-release formulation for oral administration for the treatment of hepatitis C, can achieve clinically effective and stable blood levels of the drug in the liver and portal circulation, with lower and subclinical levels of ribavirin occurring in the systemic circulation. Accordingly phases of the invention that utilize a low dose of ribavirin can achieve a systemic antiviral effect where the additive and potentiating antiviral effect of ribavirin is concentrated and retained within the liver so that the systemic side effects of ribavirin, principally anemia and its sequelae are avoided or substantially minimized. In one embodiment the low ribavirin dose is less than 400 mg/day, typically in the range of from 50 to 399 mg/day and optionally from 100 to 350 mg/day. The dose of ribavirin may be varied according to the body weight of the patient. Typically the dose will be less than 6 mg/kg/day, optionally less than 5 mg/kg/day and in certain embodiments in the range of from 1 to 5 mg/kg/day.

Certain embodiments of the invention use a high dose of ribavirin. In one embodiment the high ribavirin dose is at least 1000 mg/day, typically in the range of from 1000-1200 mg/day. The dose of ribavirin may be varied according to the body weight of the patient, with for example a high dose of ribavirin comprising 1000 mg/day in patients weighing less than 75 kilograms and comprising 1200 mg/day in patients weighing more than 75 kilograms.

Certain embodiments of the present invention provide a method of treating viral infections in a patient by co-administering a therapeutically effective amount of interferon with ribavirin that is administered as a slow release formulation, for example during the antiviral phase of embodiments of the invention. A dose of ribavirin used in certain embodiments and phases (e.g. the antiviral phase) of the invention is typically from 5 to 399 mg/day. Higher doses in the range of from 400 to 1200 mg/day may be used in other embodiments or phases (e.g. the induction and final phases), for the antiviral phase, doses of less than 400 mg/day or less than 6 mg/kg/day are typically preferred, for example a dose from 50 to 399 mg/day. The term “ribavirin analogue” as used herein includes any derivative thereof that is found to have at least 25% of ribavirin virucidal activity (see, e.g. U.S. Patent Application No. 200300044119, the contents of which are incorporated by reference). Preferably any analogue of ribavirin is administered orally as a therapeutic medicine to potentiate the antiviral effects of any form of interferon or any derivative thereof within the liver. More preferably the analogue is administered to a patient as a low-dose, slow-release formulation to deliver the drug in a liver-selective manner.

Methods for formulating the interferon and ribavirin compositions of the invention for pharmaceutical administration are known to those of skill in the art. See, for example, Remington: The Science and Practice of Pharmacy, 19^{th} Edition, Gennaro (ed.) 1995, Mack Publishing Company, Easton, Pa. Formulations to be used for in vivo administration should be sterile. This is readily accomplished by filtration through sterile filtration membranes. Acceptable carriers, excipients, or stabilizers are preferably nontoxic to cells and/or recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; and/or non-ionic surfactants such as TWEEN™, PLURONICS™ or polyethylene glycol (PEG).

Typically the therapeutic agents used in the methods of the invention combined with at pharmaceutically acceptable carrier. The term “pharmaceutically acceptable carrier” is used according to its art accepted meaning and is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration.

Therapeutic compositions of cytokines such as interferon-α and compounds such as ribavirin can be prepared by mixing the desired cytokine having the appropriate degree of purity with optional pharmaceutically acceptable carriers, excipients, or stabilizers in the form of lyophilized formulations, aqueous solutions or aqueous suspensions (see, e.g. Remington: The Science and Practice of Pharmacy Lippincott Williams & Wilkins; 21 edition (2005), and Ansel's Pharmaceutical Dosage Forms and Drug Delivery Systems Lippincott Williams & Wilkins; 8th edition (2004)). For example, pharmaceutical compositions of pegylated interferon alpha-suitable for parenteral administration may be formulated with a suitable buffer, e.g., Tris-HCl, acetate or phosphate such as dibasic sodium phosphate/monobasic sodium phosphate buffer, and pharmaceutically acceptable excipients (e.g., sucrose), carriers (e.g. human plasma albumin), toxicity agents (e.g. NaCl), preservatives (e.g. thimerosol, cresol or benzylalcohol), and surfactants (e.g. tween or polysorabates) in sterile water for injection. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances. Carriers for topical or gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols.

Solutions or suspensions used for administering a cytokine can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose.

### Typical Embodiments of the Invention

The invention disclosed herein has a number of embodiments. Typical embodiments of the invention are designed to tailor the pharmacokinetics of interferon-α delivery to a person infected with HCV using soluble, short acting interferon-α species to the time course of development of HCV resistant species that are under evolutionary pressure from an interferon-α and/or a protease inhibitor such as an inhibitor that inhibits the activity of HCV NS3-4A or a HCV polymerase (e.g. NS5B) inhibitor such as ribavirin. This is typically accomplished using mechanical infusion systems (e.g. continuous infusion pumps). Alternatively this can be accomplished via multiple daily injections of or example, a fast acting interferon-α. Without being bound by a particular scientific theory, the rationale for embodiments of the invention is the fact that there are two independent (mostly) populations of virus in patients and each population exists in two or possibly three different physiological compartments. The two types of viral populations are denoted as the wild type virus which by definition is the most competent, and the anti-viral drug induced resistant populations. The drug resistant strains are different depending on the type of target that the oral drugs are designed against, but in general the drug resistant strains are all interferon resistant.

Because of the very high replication rate of the HCV (10⁹ per day) the wild type virus rapidly re-establishes itself if the drug resistant strain is significantly reduced by interferon pressure. It is important to note that aggressive oral therapy that induces viral resistance in combination with suboptimal interferon therapy could give rise to virions that are resistant to both drugs. This situation has arisen in HIV therapies and is understandably of significant concern to medical personnel.

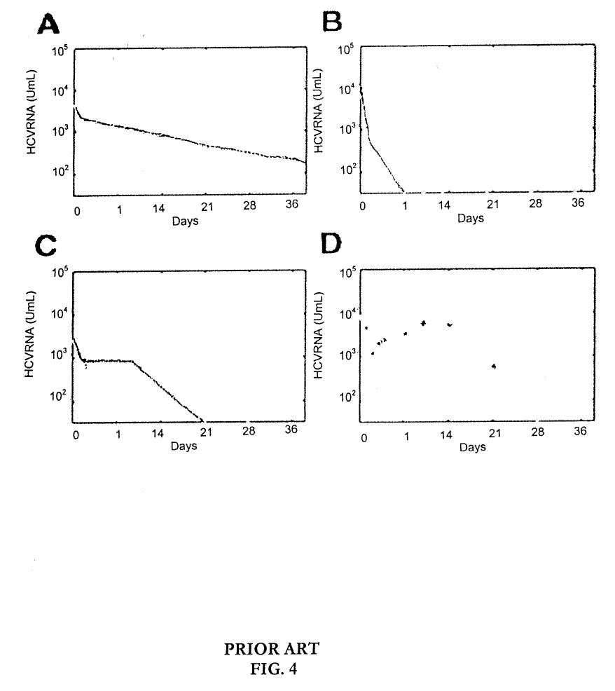

The physiological compartments that are important in treating HCV infection are demonstrated by the pharmacodynamics of interferon therapy. Plasma viral loads typically show a triphasic decay following interferon/ribavirin therapy as shown in FIG. 4. The first phase (very fast) corresponds to clearance of free virus in the blood of the patient, often from very high 10⁷ per ml levels. This phase also encompasses the blocking of de-novo infection of hepatocytes. The second phase corresponds to the net loss of infected hepatocytes. This phase is often much slower than the first phase since interferon can only act on HCV that is replicating in the cells. The third phase can be ascribed either to the immuno-modulatory effect of interferon on the non-replicating HCV in hepatocytes or alternatively to the potential mutagenic effects of ribavirin on the HCV particles. In any case, it is important to note that the highly potent anti-viral drugs need to affect all three populations of the virus.

Typical embodiments of the invention take the above-noted biological factors into account so as to pharmacokinetically control interferon therapy in combination with highly potent anti-viral drugs. In typical embodiments of the invention, the therapy comprises a triphasic drug delivery regimen. In this context, a first induction phase typically comprises high dose interferon/ribavirin therapy delivered by continuous pump based device using soluble interferon through the first phase viral decay. This phase typically ends by day 4 to day 11 depending on slope of viral load. Typically in this phase, the viral load will be measured daily. A second antiviral phase then comprises aggressive oral anti-viral therapy to increase the rate of clearance of infected cells with low level background ribavirin but no interferon therapy. The goal of this phase is to aggressively remove any infected hepatocytes and particularly any hepatocytes that are infected but not replicating (Phase 3). The goal of this phase is to remove as much wild type virus as possible, particularly removing those virus particles that are most difficult to treat with interferon. A secondary goal is to have at the end of this phase a population of only small molecule inhibitor resistant virions that are interferon sensitive. In the final phase, after the small molecules, either protease or polymerase or some other inhibitor have reduced the population of wild type virus to very low levels, then follow with 12 or more weeks of combination therapy using interferon and ribavirin again, targeting the virions that are resistant to the small molecules but sensitive to interferon. Such embodiments of the invention designed in a counter-intuitive manner to reduce the risk of developing HCV strains resistant to interferon-α and/or small molecule inhibitors such as ribavirin.

In certain embodiments of the invention, the duration of the treatment is predetermined. In an illustrative embodiment, the duration of the treatment is at least 4, 5, 6, 7, 8, 9, 10, 11 or 12 days. Alternatively, the duration of the treatment is based on a change in some factor associated with viral infection such as the levels of detectable virus in the patient. For example, in certain embodiments of the invention, the method of treating the hepatitis C viral infection results in a specific decrease in hepatitis viral levels in the human (e.g. less that 100 HCV particles per milliliter of serum). Alternatively, the duration of the treatment based upon an observation of a decrease in another factor associated with HCV infection, for example the levels of serum alanine aminotransferase in the patient.

As discussed in detail below, in the phases disclosed above, the interferon-α and/or ribavirin therapeutic agents used in the methods of the invention may be administered to the individual in combination with effective amounts of one or more other additional therapeutic agents, for example an agent that inhibits the activity of the HCV serine protease NS3-4A or the HCV non-structural protein 5B (NS5B).

A typical embodiment of the invention is a method for treating a hepatitis C virus (HCV) infected patient with a combination of interferon-α and ribavirin, the method comprising administering the interferon-α and ribavirin in sequential phases comprising: an induction phase which comprises co-administering to the patient a high dose of ribavirin and a high dose of interferon-α for at least 4 days, wherein the period of time of co-administration in the induction phase is sufficient to reduce the concentration of hepatitis C virus to less than 100 copies of HCV-RNA per ml of serum; an antiviral phase which comprises administering to the patient a low dose of ribavirin in the absence of interferon-α for at least 5 days; and a final phase which comprises co-administering to the patient a therapeutically effective amount of ribavirin and interferon-α for at least 12 weeks, wherein the period of time of co-administration in the final phase is sufficient to reduce the concentration of hepatitis C virus to less than 100 copies of HCV-RNA per milliliter of serum. Certain embodiments of the invention further comprise the step of administering to the patient at least one additional HCV antiviral compound during one or more of these sequential phases, for example VX-950, SCH 503034, R1626 or R71278. In typical embodiments of this method, the interferon-α or the ribavirin or other antiviral compound in at least one of the sequential phases is administered using a continuous infusion pump. Optionally for example, the interferon-α is administered using a continuous infusion pump during at least one of the induction or the final phases, while the ribavirin is administered orally during at least one of the sequential phases of this method.

The methods of the invention can be practiced on a wide variety of individuals infected with HCV including those previously treated for HCV infection or having a specific HCV strain. For example, some embodiments of the invention include the step of selecting the patient for treatment by identifying them as one previously treated with a course of interferon-α therapy, wherein the previous course interferon-α therapy was observed to be ineffective to treat one or more symptoms associated with the HCV infection. Other embodiments of the invention include the step of selecting the patient for treatment by identifying the patient as one infected with HCV having Genotype 1 or Genotype 1a.

As methods which use combinations of interferon-α and ribavirin in the treatment of HCV infection are well known in the art, any one of a wide variety of therapeutically effective dosage regimens for these agents can be used or adapted for use in various embodiments of the invention. In certain embodiments of the invention for example, a high dose of ribavirin is used in the induction phase, one which comprises a dose of at least 1000 mg/day. Similarly, in certain embodiments of the invention for example, a high dose of interferon is used in the induction phase, one which comprises a dose of at least 6 U/day. In some embodiments of the invention, a low dose of ribavirin is used in the antiviral phase, one which comprises a dose of less than 400 mg/day. In certain embodiments of the invention, the dose of ribavirin in the final phase comprises a dose of at least 1000 mg/day while the dose of interferon in the final phase comprises a dose of at least 6 U/day. As is known in the art, various species of interferon-α can be administered in methods designed to treat HCV infection. For example in certain embodiments of the invention, an interferon-α administered in one or more of the sequential phases is not conjugated to a polyol. In some embodiments of the invention, the interferon-α so administered comprises two interferon-α species: a first interferon-α species that is conjugated to a polyol; and a second interferon-α species that is not conjugated to a polyol. Optionally different species of interferon-α are administered in one or more of the different sequential phases of the invention.

In certain embodiments of the invention, the status of HCV in the individual is monitored during one or more of the phases of the method to obtain information useful in the tailoring the therapeutic regimen to a specific individual. Typically in certain embodiments of the invention, the initial and then changing concentrations of hepatitis C virus in the serum of the patient can be measured by a quantitative PCR method that is employed during the various phases of the method. In one illustrative embodiment, the status of HCV in the individual is monitored to determine if the period of time of co-administration in the induction phase or the final phase is sufficient to reduce the concentration of hepatitis C virus to less than 100 copies of HCV-RNA per ml of serum. In another illustrative embodiment, the status of HCV in the individual is monitored to determine if the dose of interferon-α or ribavirin or other small molecule inhibitor in a phase of the method is sufficient to reduce the concentration of hepatitis C virus to less than 100 copies of HCV-RNA per ml of serum. In yet another illustrative embodiment, the status of HCV in the individual is monitored to determine if the duration of the administration of interferon-α, ribavirin or other small molecule inhibitor during a phase of the method is sufficient to reduce the concentration of hepatitis C virus to less than 100 copies of HCV-RNA per ml of serum. Similar embodiments of the invention include the step of testing the treated patient during a phase of the method for the presence of an HCV variant that exhibits resistance to interferon-α or ribavirin (or both interferon-α and ribavirin). A variety of methods for making such measurements (e.g. those involving polynucleotide sequencing and/or HCV genotyping or species identification) which can be adapted to embodiments of the invention are known in the art (see, e.g. Jessner et al., Lancet. 2001 Oct. 13; 358(9289):1241-2).

Related embodiments of the invention include methods of inhibiting the emergence of a hepatitis C virus (HCV) having resistance to interferon-α and/or ribavirin or other small molecule inhibitor in a patient infected with wild-type hepatitis C virus and being treated with a combination of interferon-α and ribavirin. These methods comprise administering the interferon-α and ribavirin in sequential phases comprising: an induction phase which comprises co-administering to the patient a high dose of ribavirin and a high dose of interferon-α for at least 4 days, wherein the period of time of co-administration in the induction phase is sufficient to reduce the concentration of hepatitis C virus to less than 100 copies of HCV-RNA per ml of serum from the patient as measured by a quantitative PCR method; an antiviral phase which comprises administering to the patient a low dose of ribavirin in the absence of interferon-α for at least 5 days; and a final phase which comprises co-administering to the patient a high dose of ribavirin and a high dose of interferon-α for at least 12 weeks, wherein the period of time of co-administration in the final phase is sufficient to reduce the concentration of hepatitis C virus to less than 100 copies of HCV-RNA per ml of serum from the patient as measured by a quantitative PCR method such that emergence of a hepatitis C virus (HCV) having resistance to interferon-α and ribavirin in the patient infected with wild-type hepatitis C virus is inhibited. Certain embodiments of the invention further comprise the step of administering to the patient at least one additional HCV antiviral compound during one or more of the sequential phases, for example VX-950, SCH 503034, R1626 or R71278.

Optionally in such methods, the interferon-α or the ribavirin in at least one of the sequential phases is administered using a continuous infusion pump. In certain embodiments of this method, the interferon-α administered in at least one of the phases comprises two interferon-α species: a first interferon-α species that is conjugated to a polyol; and a second interferon-α species that is not conjugated to a polyol. Optionally in such methods, the patient is identified prior to administration of interferon-α or ribavirin as one infected with HCV having Genotype 1 or Genotype 1a. Typically the duration of administration or the dose of interferon-α or ribavirin during a phase of this method is adjusted based upon a determination of HCV-RNA copy number per milliliter of serum. Certain embodiments of these methods include the step of testing the treated patient during a phase of the method for the presence of an HCV variant that exhibits resistance to interferon-α or ribavirin or both interferon and ribavirin (e.g. via DNA and/or RNA sequencing and/or genotyping).

There are a number of HCV therapeutic agents known in the art in addition to ribavirin, including protease inhibitors such as Telaprevir (VX-950, Vertex) and Boceprevir (SCH 503034, Schering-Plough); as well as polymerase inhibitors such as valopicitabine (NM283, Idenix), R1626 (Roche), and HCV-796 (Viropharma) that have advanced to late-stage clinical trials for HCV infection (see, e.g. Liu-Yopung et al., AIDS Patient Care STDS. 2008; 22(6):449-57). In this context, embodiments of the invention include methods for treating a hepatitis C virus (HCV) infected patient with a combination of interferon-α and a HCV small molecule inhibitor other than or in addition to ribavirin, for example the protease inhibitor such as one that inhibits the activity of the HCV protease NS3-4A or the HCV polymerase NS5B (for example VX-950, SCH 503034, R1626 and/or R71278). A variety of such inhibitors which may be used in these methods are known in the art (see, e.g. Sheldon et al., Expert Opin Investig Drugs. 2007 August; 16(8):1171-81). One such illustrative method comprises administering the interferon-α and NS3-4A and/or NS5B inhibitor in sequential phases comprising: an induction phase which comprises co-administering to the patient a high dose of NS3-4A and/or NS5B inhibitor and a high dose of interferon-α for at least 4 days, wherein the period of time of co-administration in the induction phase is sufficient to reduce the concentration of hepatitis C virus to less than 100 copies of HCV-RNA per ml of serum; an antiviral phase which comprises administering to the patient a low dose of NS3-4A and/or NS5B inhibitor in the absence of interferon-α for at least 5 days; and a final phase which comprises co-administering to the patient a therapeutically effective amount of NS3-4A and/or NS5B inhibitor and interferon-α for at least 12 weeks, wherein the period of time of co-administration in the final phase is sufficient to reduce the concentration of hepatitis C virus to less than 100 copies of HCV-RNA per milliliter of serum.

As noted above, in some embodiments of the invention, the method further comprises administering one or more small molecule antiviral agents in addition to or in the place of ribavirin during one or more of the sequential phases of the invention. Typically one or more of these agents is administered concurrently with a low dose ribavirin during the antiviral phase. In alternative embodiments of the invention, these agents and ribavirin (and/or interferon-α) are administered sequentially. In other embodiments of the invention, these one or more antiviral agents are administered instead of ribavirin during one or more of the sequential phases of the invention. Such anti-viral agents include for example, but are not limited to, immunomodulatory agents, such as thymosin; VX-950, CYP inhibitors, amantadine, and telbivudine; Medivir's TMC435350, GSK 625433, R1626, ITMN 191, other inhibitors of hepatitis C proteases (NS2-NS3 inhibitors and NS3/NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (see, e.g., compounds of U.S. Pat. Nos. 5,807,876, 6,498,178, 6,344,465, 6,054,472, WO 97/40028, WO 98/40381, WO 00/56331 the contents of which are incorporated by reference, and mycophenolic acid and derivatives thereof, and including, but not limited to VX-497, VX-148, and/or VX-944); or combinations of any of the above. A variety of such antiviral agents that can be administered agents during one or more of the sequential phases of the invention (e.g. the antiviral phase) are known in the art, and certain illustrative examples of such are briefly discussed below. Such agents and their associated therapeutic regimens are known in the art and disclosed for example at hivandhepatitis.com and clinicaltrials.gov.

In some embodiments of the invention, an antiviral agent that is administered during one or more of the sequential phases of the invention (e.g. the antiviral phase) is VX-950 (Telaprevir). VX-950 is an orally active targeted antiviral therapy for hepatitis C virus (HCV) infection that has been shown to reduce plasma HCV RNA in patients with genotype 1 virus (see, e.g. U.S. Patent Nos. 20070218138 and 20060089385, the contents of which are incorporated by reference). In some embodiments, the dose of amorphous VX-950 can be a standard dose, e.g., about 1 g to about 5 g a day, more preferably about 2 g to about 4 g a day, more preferably about 2 g to about 3 g a day, e.g., about 2.25 g or about 2.5 g a day. For example, a dose of about 2.25 g/day of amorphous VX-950 can be administered to a patient, e.g., about 750 mg administered three times a day. Such a dose can be administered, e.g., as three 250 mg doses three times a day or as two 375 mg doses three times a day. In some embodiments, the 250 mg dose is in an about 700 mg tablet. In some embodiments, the 375 mg dose is in an about 800 mg tablet. As another example, a dose of about 2.5 g/day of amorphous VX-950 can be administered to a patient, e.g., about 1250 mg administered two times a day. As another example, about 1 g to about 2 g of amorphous VX-950 a day can be administered to a patient, e.g., about 1.35 g of amorphous VX-950 can be administered to a patient, e.g., about 450 mg administered three times a day. Vertex Pharmaceuticals Incorporated has disclosed results from an ongoing Phase 2b study evaluating Telaprevir-based treatment in patients with genotype 1 chronic hepatitis C virus (HCV) infection who did not achieve sustained virologici response (SVR) with at least one prior pegylated interferon (peg-IFN) and ribavirin (RBV) regimen. In this study, 52% (60 of 115; intent-to-treat analysis) of patients randomized to receive treatment with a 24-week Telaprevir-based regimen (12 weeks of Telaprevir in combination with peg-IFN and RBV, followed by 12 weeks of peg-IFN and RBV alone) maintained undetectable HCV RNA 12 weeks post-treatment (SVR12).

In some embodiments of the invention, an antiviral agent that is administered during one or more of the sequential phases of the invention (e.g. the antiviral phase) is SCH 503034. SCH 503034 is another hepatitis C virus (HCV) protease inhibitor (see, e.g. U.S. Patent Nos. 20070224167, 20060281688, 20070185083, 20070099825, and Sarazzin et al., Gastroenterology. 2007 April; 132(4):1270-8. Epub 2007, the contents of which are incorporated by reference). Illustrative dosing regimens for SCH 503034 include 200 mg, 300 mg, or 400 mg, 3 times daily orally. For example, genotype-1 patients in a 14-day course of treatment (5 treatment arms including 1 placebo arm), showed an HCV RNA reduction with the maximum HCV reduction of more than 2 logs in the group receiving 400 mg of SCH503034. SCH503034 was safe and well-tolerated with no serious adverse events. Schering-Plough Corporation disclosed results from an analysis of a Phase II trial of Boceprevir which showed a high rate of sustained virologic response (SVR) in patients receiving Boceprevir-based combination therapy in a study of 595 treatment-naïve patients with chronic hepatitis C virus (HCV) genotype 1. In a 48-week treatment regimen, the SVR rate at 12 weeks after the end of treatment (SVR 12) was 74 percent (ITT) in patients who received 4 weeks of PEGINTRON (peginterferon alfa-2b) and REBETOL® (ribavirin, USP) prior to the addition of Boceprevir (800 mg TID) (P/R lead-in), compared to 38 percent for patients in the control group receiving 48-weeks of PEGINTRON and REBETOL alone. Patients in the study who received 48-weeks of Boceprevir in combination with PEGINTRON and REBETOL from the beginning of treatment, (no PegIntron/ribavirin (P/R) lead-in) achieved 66 percent SVR 12. In the two 28-week Boceprevir arms of the study, SVR at 24 weeks after the end of treatment (SVR 24) was 56 percent and 55 percent for patients in the lead-in and no lead-in arms, respectively. Importantly, for patients who received the PEGINTRON and REBETOL lead in and had rapid virologic response (RVR), defined as undetectable virus (HCV-RNA) in plasma after 4 weeks of Boceprevir treatment, SVR (ITT) was 82 percent in the 28-week regimen and 92 percent in the 48 week regimen. See also, Njoroge et al. Acc Chem. Res. 2008 January; 41(1):50-9.

In other embodiments of the invention, an antiviral agent that is administered during one or more of the sequential phases of the invention (e.g. the antiviral phase) is Medivir's TMC435350 (see, e.g. the disclosure presented at the 14th International Symposium on Hepatitis C Virus and Related Viruses in Glasgow, Scotland by Simmen et al. entitled “Preclinical Characterization of TMC435350, a novel macrocyclic inhibitor of the HCV NS3/4A serine protease”, the contents of which are incorporated by reference). This disclosure demonstrates the ability of TMC435350 to reduce the amount of Hepatitis C virus replication in laboratory replicon experiments via protease inhibition. In addition, this disclosure notes that combinations of TMC435350 with interferon is also reported to enhance RNA reduction (>4 logs reduction in the replicon model), and to suppress the appearance of drug-resistance. Results presented at 43rd annual meeting of the European Association for the Study of the Liver show that TMC435350 was well tolerated during 5 days of dosing, and provoked a strong and rapid antiviral activity in genotype 1 infected individuals. See, e.g. Reesink et al., Safety of the HCV protease inhibitor TMC435350 in healthy volunteers and safety and activity in chronic hepatitis C infected individuals: a phase I study, 43rd annual meeting of the European Association for the Study of the Liver (EASL 2008), Milan, 2008.

In other embodiments of the invention, an antiviral agent that is administered during one or more of the sequential phases of the invention (e.g. the antiviral phase) is ITMN 191 (see, e.g. U.S. Patent Application No. 20050267018, the contents of which are incorporated by reference). InterMune reports that dosing in a Phase 1a single ascending-dose (SAD) trial of ITMN-191 in healthy subjects shows no serious adverse events were reported in the SAD trial. Preliminary safety data from the SAD trial provide evidence that ITMN-191 was well tolerated and safe at the doses intended for the Phase 1b multiple-ascending dose of ITMN-191. InterMune additionally reported that, based on a preliminary review of the available and still blinded clinical data from the four completed cohorts of the Phase 1b study, ITMN-191 was safe and well-tolerated.

In other embodiments of the invention, an antiviral agent that is administered during one or more of the sequential phases of the invention (e.g. the antiviral phase) is GSK 625433. A study presented at the 42nd annual meeting of the European Association for the Study of the Liver (EASL 2007) disclosed GSK625433 as a highly potent and selective inhibitor of genotype 1 HCV polymerases that is observed to be synergistic with interferon-in vitro.

In other embodiments of the invention, an antiviral agent that is administered during one or more of the sequential phases of the invention (e.g. the antiviral phase) is Taribavirin. Taribavirin (formerly known as viramidine) is an oral pro-drug of ribavirin that is less likely to cause anemia. In a study presented at the 43rd annual meeting of the European Association for the Study of the Liver (EASL 2008) in Milan, investigators disclosed results from an open-label Phase IIb trial, 278 treatment-naive patients with genotype 1 chronic hepatitis C stratified by body weight and baseline viral load and randomly assigned (1:1:1:1) to receive taribavirin at doses of 20, 25, or 30 mg/kg/day, or else weight-based ribavirin (800, 1000, 1200, or 1400 mg/day), all administered with pegylated interferon alfa-2b (PegIntron). Baseline patient characteristics were generally similar across the study arms with regard to factors predictive of treatment response.

In certain embodiments of the invention, an antiviral agent that is administered during one or more of the sequential phases of the invention (e.g. the antiviral phase) is a nucleoside having anti-HCV properties, such as those disclosed in WO 02/51425 (4 Jul. 2002), assigned to Mitsubishi Pharma Corp.; WO 01/79246, WO 02/32920, WO 02/48165 (20 Jun. 2002), and WO2005003147 (13 Jan. 2005)(including R1656, (2′R)-2′-deoxy-2′-fluoro-2′-C-methylcytidine, methylcytidine, shown as compounds 3-6 on page 77) assigned to Pharmasset, Ltd.; WO 01/68663 (20 Sep. 2001), assigned to ICN Pharmaceuticals; WO 99/43691 (2 Sep. 1999); WO 02/18404 (7 Mar. 2002), US2005/0038240 (Feb. 17, 2005) and WO2006021341 (2 Mar. 2006), including 4′-azido nucleosides such as R1626, 4′-azidocytidine, assigned to Hoffmann-LaRoche; U.S. 2002/0019363 (14 Feb. 2002); WO 02/100415 (19 Dec. 2002); WO 03/026589 (3 Apr. 2003); WO 03/026675 (3 Apr. 2003); WO 03/093290 (13 Nov. 2003); US 2003/0236216 (25 Dec. 2003); US 2004/0006007 (8 Jan. 2004); WO 04/011478 (5 Feb. 2004); WO 04/013300 (12 Feb. 2004); US 2004/0063658 (1 Apr. 2004); and WO 04/028481 (8 Apr. 2004); the content of each of which is incorporated herein by reference in its entirety. For example, patients given oral doses of R1626, (500 mg, 1500 mg, 3000 mg, 4500 mg) achieved viral load reductions of 1.2, 2.6, and 3.7 log 10 in the 100 mg, 300 mg and 4500 mg doses respectively. R1626 was generally well-tolerated with increasing adverse events at the highest dose (4500 mg). No viral resistance was found. Investigators disclosed data on R1626 at the 43rd annual meeting of the European Association for the Study of the Liver (EASL) showing that R1626 produces good response with pegylated interferon/ribavirin and has high barrier to resistance. See, e.g. Nelson et al., High End-of-Treatment Response (84%) After 4 Weeks of R1626, Peginterferon Alfa-2a (40 kd) and Ribavirin Followed By a Further 44 Weeks of Peginterferon Alfa-2a and Ribavirin. 43rd annual meeting of the European Association for the Study of the Liver (EASL 2008), Milan 2008; and Pogam et al., Low Level of Resistance, Low Viral Fitness and Absence of Resistance Mutations in Baseline Quasispecies May Contribute to High Barrier to R1626 Resistance in Vivo. 43rd annual meeting of the European Association for the Study of the Liver (EASL 2008), Milan, 2008.

In other embodiments of the invention, an antiviral agent that is administered during one or more of the sequential phases of the invention (e.g. the antiviral phase) is R71278, a polymerase inhibitor developed by Roche and Pharmasset. With R71278, there is a dose-dependent antiviral activity across all dosing arms with the 1,500 mg twice-daily arm achieving a great than 99% decrease in HCV RNA (viral load). R7128 is reported to be generally safe and well-tolerated with no serious adverse events or any dose reductions due to adverse events. Pharmasset, Inc. has disclosed results of a clinical trial evaluating R7128 1000 mg twice daily (BID) in combination with the standard of care (SOC), Pegasys plus ribavirin, in 31 treatment-naive patients chronically infected with hepatitis C virus (HCV) genotype 1. See, e.g. Lalezari et al., Inhibitor R7128 with Peg-IFN and Ribavirin: Interim Results of R7128 500 mg BID for 28 Days. 43rd annual meeting of the European Association for the Study of the Liver (EASL 2008), Milan, 2008.

Those of skill in the art will understand that there are a variety of permutations of the disclosed methods. One could for example, alter the dose or the duration of treatment depending upon aspects of HCV infection such an amount of virions eliminated and/or levels of multi-drug resistance observed in the patient. Certain of the embodiments may be adapted to the treatment of HCV infection, HIV, other “interferon-responsive diseases” such as HepB, D, leukemia, melanoma, lymphomas, Karposi's sarcoma, MS, chronic granulomatous disease, pulmonary fibrosis, and tuberculosis. In essence, embodiments of the invention can be adapted to any infection where multi-drug resistance is encountered and where a three phase approach is warranted.

In certain embodiments of the methods of the invention, the dose of the therapeutic agents(s) and route of administration may vary depending on the desired effect and/or outcome. Typically, at least one therapeutic agent is administered via a continuous infusion pump. Typically the dose and route of administration is selected to optimize the delivery a therapeutically effective amount of an agent such as interferon-α. In illustrative embodiments of the invention, an interferon-α is continuously administered in one or more phases via an infusion pump to a patient exhibiting one of more of the above signs or symptoms of viral infection in an amount and for a period of time sufficient to eliminate or at least alleviate one or more of the signs or symptoms associated with this disease.

A wide variety of continuous infusion devices known in the art can be used to deliver one or more antiviral agents to a patient infected with HCV. Continuous interferon-α administration may for example be accomplished using an infusion pump for the subcutaneous or intravenous injection at appropriate intervals, e.g. at least hourly, for an appropriate period of time in an amount which will facilitate or promote a desired therapeutic effect. Preferably the continuous infusion device used in the methods of the invention has the highly desirably characteristics that are found for example in pumps produced and sold by the Medtronic corporation. In illustrative embodiments of the invention, the cytokine is administered via an infusion pump such as a Medtronic MiniMed model 508 infusion pump. The Model 508 is currently a leading choice in insulin pump therapy, and has a long history of safety, reliability and convenience. Typically the pump includes a small, hand-held remote programmer, which enables diabetes patients to program cytokine delivery without accessing the pump itself.

Alternatively, continuous administration can by accomplished by, for example, another device known in the art such as a pulsatile electronic syringe driver (Provider Model PA 3000, Pancretec Inc., San Diego Calif.), a portable syringe pump such as the Graseby model MS1 6A (Graseby Medical Ltd., Watford, Herts England), or a constant infusion pump such as the Disetronic Model Panomat C-S Osmotic pumps, such as that available from Alza, may also be used. Since use of continuous subcutaneous injections allows the patient to be ambulatory, it is preferred over use of continuous intravenous injections.

A wide variety of formulations tailored for use with continuous infusion pumps are known in the art. For example, formulations which simulate a constant optimized dose injection, such as but not limited to short-acting unconjugated forms of interferon-α as well as long-acting interferon-α-polymer conjugates and various-sustained release formulations, are contemplated for use. Preferred routes of administration include parenteral, e.g., intravenous, intradermal, intramuscular and subcutaneous administration. Solutions or suspensions used for parenteral, intradermal, or subcutaneous application can include the following components: a sterile diluent such as water for injection, saline solution; fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates and agents for the adjustment of tonicity such as sodium chloride or dextrose. Regimens of administration may vary. Such regimens can vary depending on the severity of the disease and the desired outcome.

Following administration of a interferon-α and/or ribavirin or other therapeutic agent to a person infected with HCV, the HCV burden in the individual can be monitored in various ways well known to the skilled practitioner familiar with the hallmarks of HCV infection. In the case of chronic hepatitis infection, a therapeutically effective amount of the drug may reduce the numbers of viral particles detectable in the individual and/or relieve to some extent one or more of the signs or symptoms associated with the disorder. For example, as disclosed in detail above, in order to follow the course of hepatitis replication in subjects in response to drug treatment, hepatitis RNA may be measured in serum samples by, for example, a rt-PCR procedure such as one in which a nested polymerase chain reaction assay uses two sets of primers derived from a hepatitis genome. Farci et al., 1991, New Eng. J. Med. 325:98-104. Ulrich et al., 1990, J. Clin. Invest., 86:1609-1614. Histological examination of liver biopsy samples may then be used as a second criteria for evaluation. See, e.g., Knodell et al., 1981, Hepatology 1:431-435, whose Histological Activity Index (portal inflammation, piecemeal or bridging necrosis, lobular injury and fibrosis) provides a scoring method for disease activity.

In another embodiment of the invention, an article of manufacture (e.g. a kit) containing materials useful for the treatment of HCV infection as described above is provided. The article of manufacture can comprise a container and a label. Suitable containers include, for example, continuous infusion pumps, infusion tubing sets, catheters, bottles, vials, syringes, and test tubes. The containers may be formed from a variety of materials such as glass or plastic. The container can hold a composition (e.g. cytokine or other therapeutic composition) which is effective for treating the condition (e.g. chronic hepatitis infection) and may have a sterile access port (for example the container may be an intravenous solution bag or a vial having a stopper pierceable by a hypodermic injection needle). The label on, or associated with, the container indicates that the composition is used for treating the condition of choice. The article of manufacture may further comprise a second container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The pharmaceutical compositions useful in the methods of the invention can be included in a container, pack, or dispenser together with instructions for administration.

That result can be reduction and/or alleviation of the signs, symptoms, or causes of a disease or any other desired alteration of a biological system. For example, in a further embodiment of the invention, there are provided kits containing materials useful for treating pathological conditions with interferon. The article of manufacture comprises a container with a label. Suitable containers include, for example, bottles, vials, and test tubes. The containers may be formed from a variety of materials such as glass or plastic. The container holds a composition having an active agent which is effective for treating pathological conditions such as HCV infection. The active agent in the composition is preferably interferon-α and/or ribavirin. The label on the container indicates that the composition is used for treating pathological conditions with interferon-α and/or ribavirin.

Throughout this application, various patents, patent applications, and other publications etc. are referenced (e.g. U.S. patent No. (see, e.g. U.S. Pat. Nos. 6,172,046; 6,461,605; 6,387,365; and 6,524,570; U.S. Patent Application Nos.: 20060257365; 20070202078; 20050112093; 20050031586; 20030004119; and 20030055013). The disclosures of such publications etc. are hereby incorporated by reference herein in their entireties. The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention. However, the invention is only limited by the scope of the appended claims.

1. A method of treating a hepatitis C virus (HCV) infected human with a combination of interferon-α and ribavirin, the method comprising administering the interferon-α and ribavirin in sequential phases comprising:
(a) an induction phase which comprises co-administering to the human a high dose of ribavirin and a high dose of interferon-α for at least 4 days, wherein the period of time of co-administration in the induction phase is sufficient to reduce the concentration of hepatitis C virus to less than 100 copies of HCV-RNA per ml of serum;
(b) an antiviral phase which comprises administering to the human a low dose of ribavirin in the absence of interferon-α for at least 5 days; and
(c) a final phase which comprises co-administering to the human a therapeutically effective amount of ribavirin and interferon-α for at least 12 weeks, wherein the period of time of co-administration in the final phase is sufficient to reduce the concentration of hepatitis C virus to less than 100 copies of HCV-RNA per milliliter of serum.

2. The method of claim 1, further comprising administering the interferon-α or the ribavirin in at least one of the sequential phases using a continuous infusion pump.

3. The method of claim 1, further comprising administering the interferon-α using a continuous infusion pump during at least one of the induction or the final phases and administering the ribavirin orally during at least one of the sequential phases.

4. The method of claim 1, further comprising selecting the human for treatment by identifying the human as one previously treated with a course of interferon-α therapy, wherein the previous course interferon-α therapy was observed to be ineffective to treat one or more symptoms associated with the HCV infection.

5. The method of claim 1, further comprising selecting the human for treatment by identifying the human as one infected with HCV having Genotype 1 or Genotype 1a.

6. The method of claim 1, wherein the high dose of ribavirin in the induction phase comprises a dose of at least 1000 mg/day and the high dose of interferon in the induction phase comprises a dose of at least 6 IU/day.

7. The method of claim 1, wherein the low dose of ribavirin in the antiviral phase comprises a dose of less than 400 mg/day.

8. The method of claim 1, wherein the dose of ribavirin in the final phase comprises a dose of at least 1000 mg/day and the dose of interferon in the final phase comprises a dose of at least 6 IU/day

9. The method of claim 1, wherein the interferon-α is not conjugated to a polyol.

10. The method of claim 1, wherein the interferon-α so administered comprises two interferon-α species: a first interferon-α species that is conjugated to a polyol; and a second interferon-α species that is not conjugated to a polyol.

11. The method of claim 1, wherein the period of time of co-administration in the induction phase or the final phase is sufficient to reduce the concentration of hepatitis C virus to less than 50 copies of HCV-RNA per ml of serum of the human as measured by a quantitative PCR method.

12. The method of claim 1, further comprising adjusting a dose of interferon-α or ribavirin in a phase of the method based upon a determination of HCV-RNA copy number per milliliter of serum.

13. The method of claim 1, further comprising adjusting the duration of the administration of interferon-α or the ribavirin during a phase of the method based upon a determination of HCV-RNA copy number per milliliter of serum.

14. The method of claim 1, further comprising the step of testing the treated human during a phase of the method for the presence of an HCV variant that exhibits resistance to interferon-α or ribavirin.

15. The method of claim 1, further comprising the step of administering to the human at least one additional HCV antiviral compound during one or more of the sequential phases, wherein the compound is selected from the group consisting of VX-950, SCH 503034, R1626 and R71278.

16. A method of inhibiting the emergence of a hepatitis C virus (HCV) having resistance to interferon-α and ribavirin in a human infected with wild-type hepatitis C virus and being treated with a combination of interferon-α and ribavirin, the method comprising administering the interferon-α and ribavirin in sequential phases comprising:
(a) an induction phase which comprises co-administering to the human a high dose of ribavirin and a high dose of interferon-α for at least 4 days, wherein the period of time of co-administration in the induction phase is sufficient to reduce the concentration of hepatitis C virus to less than 100 copies of HCV-RNA per ml of serum from the human as measured by a quantitative PCR method;
(b) an antiviral phase which comprises administering to the human a low dose of ribavirin in the absence of interferon-α for at least 5 days; and
(c) a final phase which comprises co-administering to the human a high dose of ribavirin and a high dose of interferon-α for at least 12 weeks, wherein the period of time of co-administration in the final phase is sufficient to reduce the concentration of hepatitis C virus to less than 100 copies of HCV-RNA per ml of serum from the human as measured by a quantitative PCR method;
such that emergence of a hepatitis C virus (HCV) having resistance to interferon-α and ribavirin in the human infected with wild-type hepatitis C virus is inhibited.

17. The method of claim 16, further comprising administering the interferon-α or the ribavirin in at least one of the sequential phases using a continuous infusion pump.

18. The method of claim 16, wherein the interferon-α administered in at least one of the phases comprises two interferon-α species: a first interferon-α species that is conjugated to a polyol; and a second interferon-α species that is not conjugated to a polyol.

19. The method of claim 16, further comprising selecting the human for treatment by identifying the human as one infected with HCV having Genotype 1 or Genotype 1a.

20. The method of claim 16, further comprising adjusting the duration of administration or the dose of interferon-α or ribavirin during a phase of the method based upon a determination of HCV-RNA copy number per milliliter of serum.

21. The method of claim 16, further comprising the step of testing the treated human during a phase of the method for the presence of an HCV variant that exhibits resistance to interferon-α or ribavirin or both interferon and ribavirin.

22. The method of claim 16, further comprising the step of administering to the human at least one additional HCV antiviral compound during one or more of the sequential phases, wherein the compound is selected from the group consisting of VX-950, SCH 503034, R1626 and R71278.
